(19) United States
(12) Patent Application Publication
(10) Pub. No.: US 2022/0047574 A1
(43) Pub. Date: Feb. 17, 2022

(54) CRENOLANIB FOR TREATING PDGFR ALPHA MUTATED PROLIFERATIVE DISORDERS

(71) Applicant: Arog Pharmaceuticals, Inc., Plano, TX (US)

(72) Inventor: Vinay K. Jain, Dallas, TX (US)

(21) Appl. No.: 17/320,316

(22) Filed: May 14, 2021

Related U.S. Application Data
(60) Provisional application No. 63/066,195, filed on Aug. 15, 2020.

(51) Int. Cl. A61K 31/4709

(52) U.S. Cl. CPC A61K 31/4709

(57) ABSTRACT

The present invention includes methods for treating a PDGFRα mutated proliferative disorder in a subject relapsed/refractory to prior tyrosine kinase inhibitor therapy comprising administering to the subject a therapeutically effective of crenolanib,

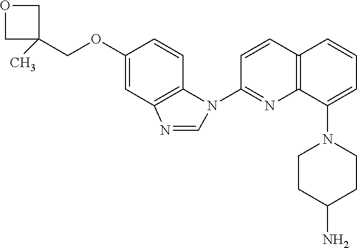 [SMILES: CC1(COc2ccc3c(c2)ncn3-c2ccc3cccc(N4CCC(N)CC4)c3n2)COC1]

wherein the subject is relapsed/refractory to prior tyrosine kinase inhibitor therapy due to resistance mutations or wherein the subject discontinued prior tyrosine kinase inhibitory therapy due to toxicities.

### CROSS-REFERENCE TO RELATED APPLICATIONS

This application claims priority to U.S. Provisional Application Ser. No. 63/066,195, filed Aug. 15, 2020, the entire contents of which are incorporated herein by reference.

### TECHNICAL FIELD OF THE INVENTION

The present invention is directed to the use of crenolanib, or salts thereof, as a single agent or in combination with another pharmaceutical agent for the treatment of cancer, and to methods for treating animals suffering from cancer.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with platelet-derived growth factor receptor (PDGFR) tyrosine kinases and their role in cancer.

The PDGFR tyrosine kinases, PDGFR alpha and PDGFR beta, are involved in a number of normal physiological processes. However, dysregulation of these signaling pathways through activating mutations is associated with the development of proliferative diseases such as cancer. One such mutation, PDGFRA-D842V is the second most frequently found mutation in gastrointestinal stromal tumors (GIST) after mutations in KIT, present in up to 14% of GIST (Szucs et al., 2017).

Patients with metastatic or advanced unresectable GIST have a very poor prognosis due to few surgical options and the ineffectiveness of conventional chemotherapy and radiotherapy on tumor burden (Blackstein et al., 2006; Casali et al., 2008; DeMatteo, Heinrich, El-Rifai, & Demetri, 2002). Research and discovery dating as early as 2001 revealed the molecular pathophysiology of GIST, which revolutionized treatment options and clinical outcomes with the development of imatinib mesylate, a type II tyrosine kinase inhibitor (TKI) with activity against KIT and PDGFR alpha tyrosine kinases. Imatinib mesylate has significantly improved outcomes for patients and is the standard of care for first-line treatment of GIST (Serrano & George, 2014). Depending on mutation status, however, the response to imatinib varies. For example, patients with KIT exon 11 mutation have a greater chance of benefit than patients that harbor a KIT mutation in exon 9 (Marrari, Wagner, & Hornick, 2012). Molecular characterization of GIST also revealed other driver mutations associated with poor prognosis in metastatic or advanced GIST.

Unfortunately, patients harboring D842V mutations in the PDGFRA gene do not respond to imatinib at all. Type II TKIs, such as imatinib, are only capable of binding to the inactive conformation of the PDGFRA receptor tyrosine kinase and have little to no activity against D842V-mutated PDGFRA, a highly activating mutation that induces constitutive PDGFRA-activation in the absence of ligand (Corless, Barnett, & Heinrich, 2011). Despite remarkable advances in the treatment of advanced GIST, resistance in specific molecular subtypes, such as PDGFRA-D842V, remains a problem. Analysis of kinase genotype and clinical outcome correlation in a Phase III (SWOG 50033/CALGB 150105) trial of imatinib for advanced GIST revealed that PDGFRA-D842V patients had progression free survival (PFS) of <2 months (M. C. Heinrich, Owzar, et al., 2008). Analysis of data from institutional databases also showed PDGFRA D842V-mutated GIST was unresponsive to imatinib, with a median PFS of 2.8 months with first-line imatinib and 2.1 months with second-line imatinib (Cassier et al., 2012). Another study bolstered these findings, showing the inefficacy of first-line imatinib in D842V-mutated GIST, with a median overall survival (OS) of 25.5 months in D842V-mutated GIST versus 59.8 months in non-D842V mutated GIST (Yoo et al., 2016).

More recently, sunitinib and regorafenib were approved for the treatment of advanced GIST following resistance or intolerance to imatinib as 2nd or 3rd line therapy, respectively (Serrano & George, 2014). A correlative study found that sunitinib treatment after imatinib failure provided clinical benefit to the three most common primary GIST genotypes (KIT exon 9, KIT exon 11, KIT/PDGFRA wild-type) but patients with PDGFRA mutations experienced no clinical benefit to secondary sunitinib treatment (M. C. Heinrich, Maki, et al., 2008). Metastatic disease with acquired drug resistance is hypothesized to be the result of secondary, imatinib-resistant mutations in KIT or PDGFRA (Antonescu et al., 2005). Regorafenib treatment is indicated only for patients with disease progression on imatinib and sunitinib. However, both sunitinib and regorafenib, like imatinib, have shown no activity against the D842V mutation in PDGFRA-mutated GIST. These data highlight the need for more PDGFR-specific inhibitors, especially in advanced PDGFRA D842V-mutated GIST, which currently has no effective and approved treatment regimen.

In 2020, avapritinib was approved for use in GIST harboring PDGFRA exon 18 mutations, including PDGFRA-D842V. While the efficacy of avapritinib for this imatinib resistant population provides high response rates and promising progression free survival (Michael C. Heinrich et al., 2020), serious toxicities including: intracranial hemorrhage and central nervous system effects such as cognitive impairment, mood and sleep disorders, and hallucinations, have been reported in a significant number of patients (FDA, 2020). Other adverse events requiring treatment discontinuation included encephalopathy, dizziness, fatigue, vomiting, abdominal pain, anemia, sepsis, and acute kidney injury. Overall, 16% of patients included in the United States Food and Drug Administration analysis completed as part of the approval process for avapritinib discontinued treatment due to toxicities. Furthermore, fatal adverse reactions, including sepsis and tumor hemorrhage, occurred in 3.6% of patients (FDA, 2020).

In addition to the significant safety concerns patients have experienced on avapritinib, the development of secondary mutations that confer resistance is a known complication in the use of tyrosine kinase inhibitors. Imatinib, the first TKI approved for use in any cancer, was first approved for use in BCR-ABL positive chronic myelogenous leukemia, and point mutations in ABL conferring resistance to imatinib treatment were reported in the literature as early as 2002 (Hochhaus et al., 2002). Mutations at amino acid residues V658, E675, Y676, G680, and G741 have been reported in patients resistant to avapritinib. Other mutations within the tyrosine kinase domains or the hinge region and kinase insert domain may also confer resistance to avapritinib. These mutations may be de novo, in other words present at diagnosis and causing primary refractory disease, or acquired after avapritinib treatment, leading to resistance and disease progression while on treatment.

Given the relatively high percentage of patients who discontinue avapritinib due to toxicities, as well as the number of patients with mutations within the PDGFRA gene which confer resistance to avapritinib and other TKIs, there remains an unmet need in patients suffering from PDGFRA mutated proliferative disorders.

### SUMMARY OF THE INVENTION

In one embodiment, the present invention includes a method of inhibiting or reducing mutant PDGFRα tyrosine kinase activity or expression in a subject suffering from a proliferative disorder or proliferative disease comprising: obtaining a tumor sample from the subject; measuring expression of a mutated PDGFRα or a constitutively active PDGFRα mutant; and administering to the subject a therapeutically effective amount of crenolanib or a pharmaceutically acceptable salt thereof wherein the crenolanib or salt thereof reduces the proliferative disorder burden or prevents proliferative disease progression. In one aspect, the subject is relapsed/refractory to a prior tyrosine kinase inhibitor. In another aspect, the subject has been provided a prior tyrosine kinase inhibitor selected from imatinib or avapritinib. In another aspect, the subject has a PDGFRα mutation that is resistant to avapritinib. In another aspect, the PDGFRα mutation is selected from a missense mutation at D68, D135, D173, E229, C235, E262, T276, E289, K385, T440, A498, V561, R588, G608, N659, E675, Y676, S695, G741, G829, R841, I843, D846, Y849, N848, A1014, or D1071 present alone or in combination with a D842 missense mutation. In another aspect, the PDGFRα mutation is selected from inframe deletions or insertions at amino acid residues R560-V561, R560-S564, E561-R562, S566-571, I843, D842-H845, or H845-5847 present alone or in combination with a D842 missense mutation. In another aspect, the proliferative disorder is selected from at least one of a gastrointestinal stromal tumor, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. In another aspect, the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day. In another aspect, the therapeutically effective amount of crenolanib or the pharmaceutically acceptable salt thereof is administered at least one of continuously, intermittently, systemically, or locally. In another aspect, the therapeutically effective amount of crenolanib or the pharmaceutically acceptable salt thereof is administered orally, intravenously, or intraperitoneally. In another aspect, the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, or crenolanib succinate. In another aspect, the therapeutically effective amount of crenolanib or the pharmaceutically acceptable salt thereof is administered up to three times or more a day for as long as the subject is in need of treatment for the proliferative disorder. In another aspect, the therapeutically effective amount of crenolanib or the pharmaceutically acceptable salt thereof is provided at least one of sequentially or concomitantly, with another pharmaceutical agent.

In another embodiment, the present invention includes a method of inhibiting or reducing mutant PDGFRα tyrosine kinase activity or expression in a subject suffering from a proliferative disorder or proliferative disease comprising; identifying that the subject discontinued a first tyrosine kinase inhibitor therapy due to toxicity or toxicities; obtaining a tumor sample from the subject; measuring expression of a mutated PDGFRα or a constitutively active PDGFRα mutant; and if the subject has the mutated PDGFRα or constitutively active PDGFRα mutant, administering to the subject a therapeutically effective amount of crenolanib or a pharmaceutically acceptable salt thereof wherein the crenolanib or salt thereof reduces the proliferative disorder burden or prevents proliferative disease progression. In one aspect, the toxicity or toxicities requiring discontinuation of the first tyrosine kinase inhibitor therapy include one or more of intracranial hemorrhage, central nervous system toxicity, fatigue, abdominal pain, vomiting, sepsis, anemia, acute kidney injury, and encephalopathy. In another aspect, the intracranial hemorrhage includes one or more of subdural hematoma, cerebral hemorrhage, or other intracranial hemorrhage.

In another aspect, the central nervous system toxicity includes one or more of cognitive impairment, dizziness, sleep disorders, mood disorders, and hallucinations. In another aspect, the cognitive impairment includes one or more of memory impairment, cognitive disorder, confused state, disturbance in attention, amnesia, mental impairment, mental status changes, dementia, abnormal thinking, mental disorders, and retrograde amnesia. In another aspect, the proliferative disorder is selected from at least one of a gastrointestinal stromal tumor, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. In another aspect, the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day. In another aspect, the therapeutically effective amount of crenolanib or the pharmaceutically acceptable salt thereof is administered at least one of continuously, intermittently, systemically, or locally. In another aspect, the therapeutically effective amount of crenolanib or the pharmaceutically acceptable salt thereof is administered orally, intravenously, or intraperitoneally. In another aspect, the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, and crenolanib succinate. In another aspect, the therapeutically effective amount of crenolanib or the pharmaceutically acceptable salt thereof is administered up to three times or more a day for as long as the subject is in need of treatment for the proliferative disorder. In another aspect, the therapeutically effective amount of crenolanib or the pharmaceutically acceptable salt thereof is provided at least one of sequentially or concomitantly, with another pharmaceutical agent.

In another embodiment, the present invention includes a method for treating a patient is suffering from a proliferative disorder or a proliferative disease, the method comprising the steps of: determining whether the patient has increased PDGFRα tyrosine kinase activity by: obtaining or having obtained a biological sample from the patient; and performing or having performed an assay on the biological sample to determine if the patient has a gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity; treating the patient with a first tyrosine kinase inhibitor (TKE); and if the patient experiences a toxicity or toxicities to the first TKI and the patient has the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, then discontinue administering the first TKI and internally administering crenolanib to the patient in an effective amount, or if the patient does not experience a toxicity or toxicities to the first TKI and the patient has the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, but the proliferative disorder or a proliferative disease progresses then discontinue administering the first TKI and internally administering crenolanib to the patient in an effective amount, or if the patient has the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, and has proliferative disorder or proliferative disease progression then internally administering crenolanib to the patient in an effective amount, wherein a risk of toxicity, toxicities or proliferative disorder or proliferative disease progression for a patient having the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, is lower following the internal administration of crenolanib. In one aspect, the toxicity or toxicities requiring discontinuation of the first tyrosine kinase inhibitor therapy include one or more of intracranial hemorrhage, central nervous system toxicity, fatigue, abdominal pain, vomiting, sepsis, anemia, acute kidney injury, and encephalopathy. In another aspect, the intracranial hemorrhage includes one or more of subdural hematoma, cerebral hemorrhage, or other intracranial hemorrhage. In another aspect, the central nervous system toxicity includes one or more of cognitive impairment, dizziness, sleep disorders, mood disorders, and hallucinations. In another aspect, the cognitive impairment includes one or more of memory impairment, cognitive disorder, confused state, disturbance in attention, amnesia, mental impairment, mental status changes, dementia, abnormal thinking, mental disorders, and retrograde amnesia. In another aspect, the proliferative disorder is selected from at least one of a gastrointestinal stromal tumor, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. In another aspect, the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day. In another aspect, the therapeutically effective amount of crenolanib or the pharmaceutically acceptable salt thereof is administered at least one of continuously, intermittently, systemically, or locally. In another aspect, the therapeutically effective amount of crenolanib or the pharmaceutically acceptable salt thereof is administered orally, intravenously, or intraperitoneally. In another aspect, the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, and crenolanib succinate. In another aspect, the therapeutically effective amount of crenolanib or the pharmaceutically acceptable salt thereof is administered up to three times or more a day for as long as the subject is in need of treatment for the proliferative disorder. In another aspect, the therapeutically effective amount of crenolanib or the pharmaceutically acceptable salt thereof is provided at least one of sequentially or concomitantly, with another pharmaceutical agent. In another aspect, the PDGFRα mutation is selected from a missense mutation at D68, D135, D173, E229, C235, E262, T276, E289, K385, T440, A498, V561, R588, G608, N659, E675, Y676, S695, G741, G829, R841, I843, D846, Y849, N848, A1014, or D1071 present alone or in combination with a D842 missense mutation. In another aspect, the PDGFRα mutation is selected from inframe deletions or insertions at amino acid residues R560-V561, R560-S564, E561-R562, S566-571, I843, D842-H845, or H845-S847 present alone or in combination with a D842 missense mutation.

### BRIEF DESCRIPTION OF THE DRAWINGS

For a more complete understanding of the features and advantages of the present invention, reference is now made to the detailed description of the invention along with the accompanying figures and in which:

For a more complete understanding of the features and advantages of the present invention, reference is now made to the detailed description of the invention along with the accompanying figures and in which:

FIG. 1 shows the dose-response curve for IC50 determination of the besylate salt of the present invention for wildtype PDGFRα. The activity of the besylate salt of crenolanib is plotted against the corresponding molar concentration in log 10 scale.

FIG. 2 shows the dose-response curve for IC50 determination of the besylate salt of the present invention for the PDGFRα-D842V. The activity of the besylate salt of crenolanib is plotted against the corresponding molar concentration in log 10 scale.

FIG. 3 shows the dose-response curve for IC50 determination of the besylate salt of the present invention for the PDGFRα-T6741. The activity of the besylate salt of crenolanib is plotted against the corresponding molar concentration in log 10 scale.

FIG. 4 shows the dose-response curve for IC50 determination of the besylate salt of the present invention for the PDGFRα-V561D. The activity of the besylate salt of crenolanib is plotted against the corresponding molar concentration in log 10 scale.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The present invention is directed to the administration of crenolanib, or a pharmaceutically acceptable salt thereof, to subjects suffering from cancer in order to treat the cancer, prevent reoccurrence of the cancer, and/or prevent worsening of the cancer.

Crenolanib is an orally bioavailable TKI, targeting both alpha and beta receptor types. It is significantly more selective for PDGFR than other kinases, including c-KIT, VEGFR2, TIE2, FGFR2, EGFR, erbB2, and SRC (Lewis et al., 2009). As a type I TKI, it binds to both the active and inactive conformations of the kinase. Importantly, crenolanib shows clinical and preclinical activity against imatinib resistant PDGFRA exon 18 mutations, including D842V, D8421, and D842Y (M. C. Heinrich et al., 2012). In cell lines overexpressing D842V-PDGFRA, crenolanib blocks phosphorylation of PDGFRα at nanomolar concentrations. Furthermore, crenolanib has shown activity against resistance conferring mutations within PDGFRA. As such, crenolanib is ideally suited for the treatment of patients suffering from PDGFRA proliferative disorders who have discontinued treatment with other TKIs due to either toxicity or resistance conferring secondary mutations.

The present invention comprises methods of inhibiting mutant PDGFRα in a cell or a subject, or to treat disorders related to PDGFRα kinase activity or expression in a subject. In one embodiment, the present invention provides a method for reducing or inhibiting the kinase activity of mutant PDGFRα in a subject comprising the step of administering a compound of the present invention to the subject. In other embodiments, the present invention provides therapeutic methods for treating a subject with a cell proliferative disorder driven by aberrant kinase activity of mutant PDGFRα. The present invention also provides methods for treating a patient suffering from a proliferative disorder that is relapsed/refractory to a prior tyrosine kinase inhibitor.

As used herein, the term “subject” refers to an animal, such as a mammal or a human, who has been the object of treatment, observation or experiment.

As used herein, the term “contacting” refers to the addition of Crenolanib or pharmaceutically acceptable salt(s) thereof, to cells such that the compound is taken up by the cell.

As used herein, the term “therapeutically effective amount” refers to an amount of Crenolanib or pharmaceutically acceptable salt(s) thereof, that elicits the biological or medicinal response in a subject that is being sought by a researcher, veterinarian, medical doctor or other clinician, which includes alleviation of the symptoms of the disease or the disorder being treated, reduction in the burden of the proliferative disorder (such as reduction in tumor size), and/or increase in progression-free or overall survival including prolonged stable disease. Methods for determining therapeutically effective doses for pharmaceutical compositions comprising a compound of the present invention are known in the art.

As used herein, the term “composition” is intended to encompass a product comprising the specified ingredients in the specified amounts, as well as any product which results, directly or indirectly, from combinations of the specified ingredients in the specified amounts.

As used herein, the terms “disorder related to mutant PDGFRα”, or “mutant PDGFRα driven cell proliferative disorder” includes diseases associated or implicating mutant PDGFRα activity, for example, mutations leading to constitutive activation of PDGFRα.

As used herein, the term “cell proliferative disorders” refers to excess cell proliferation of one or more subset of cells in a multicellular organism resulting in harm (i.e., discomfort or decreased life expectancy) to the multicellular organism. Cell proliferative disorders can occur in different types of animals and humans. Examples of cell proliferative disorders are gastrointestinal stromal tumor (GIST), leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy.

As used herein, the term “relapsed/refractory” refer(s) to a subject that was previously administered a pharmaceutical agent in order to treat a proliferative disease, but either did not respond to treatment (refractory), or progressed after initially responding (relapsed).

Detection of the mutated PDGFRα can be performed using any suitable method known in the art. For example, detection of gene mutations can be accomplished by detecting nucleic acid molecules (such as DNA) using nucleic acid amplification methods (such as RT-PCR) or high-throughput sequencing (i.e. “next-generation sequencing” (NGS)). For example, NGS platforms such as Illumina may be used to determine the exact genetic sequence of specific genes, or portions of genes, of interest. In brief, DNA from a tumor sample is fragmented, ligated with the appropriate primers and adaptors, and amplified using PCR during “library preparation”. The prepared libraries are then sequenced using one of a number of commercially available systems which generates the sequence of the chosen target genes, all exomes, or the entire genome. The sequences are then analyzed using commercial available software, which aligns the tumor sample sequence to the known sequence of the genes of interest and performs a variant calling step, which identifies differences at the DNA level in the tumor sample and determines if such mutations would result in alteration of the amino acid sequence in the translated protein. Using these systems, a person of skill in the art can determine if a subject has one of the identified mutations with in the PDGFRA gene.

As used herein, the term “missense mutation” refers to alterations in the genetic sequence of the PDGFRA gene that results in the substitution of one amino acid for a different amino acid when the sequence is translated into a protein.

As used herein, the term “missense mutation” refers to a nucleotide mutation in the DNA sequence that results in an amino acid substitution at the protein level.

As used herein, the term “in frame deletion” refers to the loss of nucleotides at the DNA level in which the number of nucleotides deleted is a multiple of three, which results in a loss of amino acids at the protein level but does not shift the reading frame of the gene.

As used herein, the terms “resistance mutations”, or “mutations conferring resistance”, or “secondary mutations” refer to mutations other than D842V within the PDGFRA gene that are not sensitive to avapritinib or other TKIs, other than the present invention. In other words, these mutations, whether present alone or in combination with D842V, retain kinase activity when treated with avapritinib or other TKIs but are inhibited by the present invention. Non-limiting examples of resistance mutations are missense mutations at amino acid residues D68, D135, D173, E229, C235, E262, T276, E289, K385, T440, A498, V561, R588, G608, N659, E675, Y676, S695, G741, G829, R841, I843, D846, Y849, N848, A1014, or D1071. Further mutations include inframe deletions at amino acid residues R560-V561, R560-S564, E561-R562, S566-571, I843, D842-H845, or H845-S847.

Additional mutations within the juxtamembrane domain, tyrosine kinase domains, hinge region, and kinase insert domain, (i.e., between amino acid residues 550 and 1089), are also included within the scope of the present invention.

In another embodiment of this aspect, the present invention provides methods for treating a patient suffering from a proliferative disorder wherein the subject discontinued treatment with another pharmaceutical agent due to toxicities.

As used herein, the terms “toxicity” or “toxicities” refers to side effects, adverse events, or adverse reactions experienced by a subject while receiving a particular pharmaceutical agent. In particular, those side effects, adverse events, or adverse reactions where are determined to be related to the pharmaceutical agent and which diminish or disappear when the pharmaceutical agent is discontinued. Examples of toxicities with the TKI of the prior art include, e.g., intracranial hemorrhage and central nervous system effects such as cognitive impairment, mood and sleep disorders, hallucinations, encephalopathy, dizziness, fatigue, vomiting, abdominal pain, anemia, sepsis, and acute kidney injury. In one aspect, the present invention does not cause one or more of these toxicities.

In one embodiment, the present invention therapeutically effective amounts of the compound having Formula I:

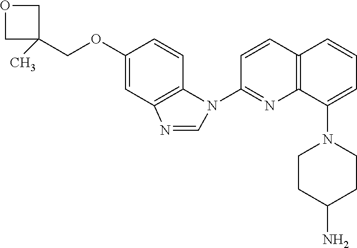 [SMILES: CC1(COc2ccc3c(c2)ncn3-c2ccc3cccc(N4CCC(N)CC4)c3n2)COC1]

or a pharmaceutically acceptable salt or solvate thereof, in a therapeutically effective amount against a proliferative disease is selected from at least one of gastrointestinal stromal tumor, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. Pharmaceutically acceptable salts such as hydrochloride, phosphate and lactate are prepared in a manner similar to the benzenesulfonate salt and are well known to those of moderate skill in the art. Pharmaceutically acceptable salts such as hydrochloride, phosphate and lactate are prepared in a manner similar to the benzenesulfonate salt and are well known to those of moderate skill in the art. The following representative compounds of the present invention are for exemplary purposes only and are in no way meant to limit the invention, including Crenolanib as Crenolanib Besylate, Crenolanib Phosphate, Crenolanib Lactate, Crenolanib Hydrochloride, Crenolanib Citrate, Crenolanib Acetate, Crenolanib Toluenesulphonate and Crenolanib Succinate.

Compounds of the present invention may be administered to a subject systemically, for example, orally, intravenously, subcutaneously, intramuscular, intradermal or parenterally. The compounds of the present invention can also be administered to a subject locally.

Compounds of the present invention may be formulated for slow-release or fast-release with the objective of maintaining contact of compounds of the present invention with targeted tissues for a desired range of time.

Compositions suitable for oral administration include solid forms, such as pills, tablets, caplets, capsules, granules, and powders, liquid forms, such as solutions, emulsions, and suspensions. Forms useful for parenteral administration include sterile solutions, emulsions and suspensions.

The daily dosage of the compounds of the present invention may be varied over a wide range from 50 to 500 mg per adult human per day. For oral administration, the compositions are preferably provided in the form of tablets containing 20 and 100 milligrams. The compounds of the present invention may be administered on a regimen up to three times or more per day. Preferably three times per day. Optimal doses to be administered may be determined by those skilled in the art, and will vary with the compound of the present invention used, the mode of administration, the time of administration, the strength of the preparation, the details of the disease condition. Factors associated with patient characteristics, such as age, weight, and diet will call for dosage adjustments. In other examples, the daily dosage of the compounds of the present invention may be varied over a wide range from 15 to 500, 25 to 450, 50 to 400, 100 to 350, 150 to 300, 200 to 250, 15, 25, 50, 75, 100, 150, 200, 250, 300, 400, 450, or 500 mg per day. The compounds of the present invention may be administered on a daily regimen, once, twice, three or more times per day. Optimal doses to be administered may be determined by those skilled in the art, and will vary with the compound of the present invention used, the mode of administration, the time of administration, the strength of the preparation, the details of the disease condition. One or more factors associated with subject characteristics, such as age, weight, and diet will call for dosage adjustments. Techniques and compositions for making useful dosage forms using the Crenolanib are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); relevant portions incorporated herein by reference.

A dosage unit for use of Crenolanib, may be a single compound or mixtures thereof with other compounds, e.g., a potentiator. The compounds may be mixed together, form ionic or even covalent bonds. The compounds of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the Crenolanib of the present invention to a patient in need of therapy.

The Crenolanib is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices Depending on the best location for administration, the Crenolanib may be formulated to provide, e.g., maximum and/or consistent dosing for the particular form for oral, rectal, topical, intravenous injection or parenteral administration. While the Crenolanib may be administered alone, it will generally be provided in a stable salt form mixed with a pharmaceutically acceptable carrier. The carrier may be solid or liquid, depending on the type and/or location of administration selected.

Preparation of the compounds of the present invention. General synthetic methods, which may be referred to for preparing the compounds of Formula I are provided in U.S. Pat. No. 5,990,146 (issued Nov. 23, 1999) (Warner-Lambert Co.) and PCT published application numbers WO 99/16755 (published Apr. 8, 1999) (Merck & Co.) WO 01/40217 (published Jul. 7, 2001) (Pfizer, Inc.), US Patent Application No. US 2005/0124599 (Pfizer, Inc.) and U.S. Pat. No. 7,183,414 (Pfizer, Inc.), relevant portions incorporated herein by reference.

Pharmaceutically acceptable salts such as hydrochloride, phosphate and lactate are prepared in a manner similar to the benzenesulfonate salt and are well known to those of moderate skill in the art. The following representative compounds of the present invention are for exemplary purposes only and are in no way meant to limit the invention.

Biological Activity

In vitro Assays. The following representative in vitro assays were performed in determining the PDGFRAα biological activity of the present invention. These are given to illustrate the invention in a non-limiting fashion.

Inhibition of mutant PDGFRα enzyme activity exemplifies the specific inhibition of the mutant PDGFRα enzyme and cellular processes that are dependent on mutant PDGFRα activity. All of the examples herein show significant and specific inhibition of mutant PDGFRα kinase and PDGFRα-dependent cellular responses.

Direct enzyme phosphorylation assay. The Reaction Biology HotSpot Kinase assay was used to screen the present invention against a panel of normal PDGFRα and mutated PDGFRα kinases. For assays of both kinases, the PDGFRα enzyme was prepared in base reaction buffer (20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO). The reaction was initiated by the addition of ³³P-ATP (10 μCi/μL) into the mixture. The reaction mixture was incubated for 120 minutes at room temperature. Radioactivity was detected by filter-binding method, and kinase activity expressed as the percent remaining kinase activity in test samples compared to vehicle reactions. IC50 values and curve fitting were obtained using Prism (GraphPad Software).

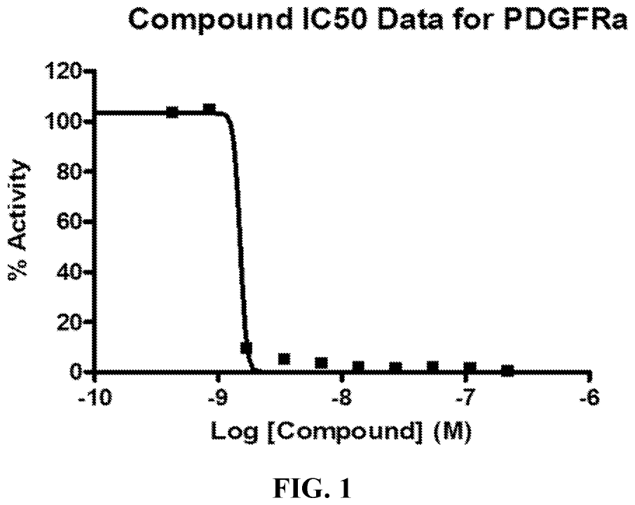

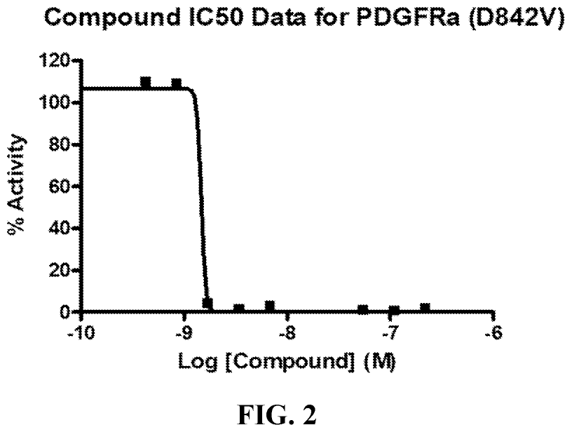

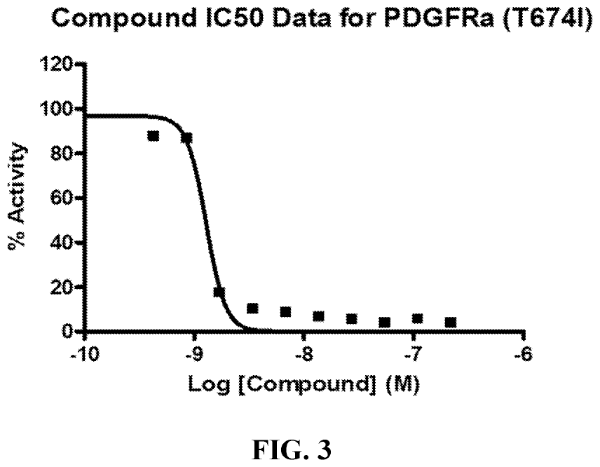

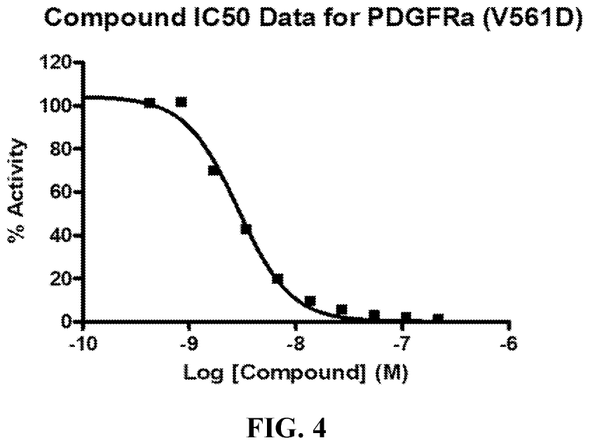

The activity of the besylate salt of the present invention was determined using a direct enzymatic Reaction Biology HotSpot Kinase assay. All IC50 values are presented in nanomolar concentration. In the direct enzymatic measurement assay, the IC50 of the besylate salt of the current invention against the various PDGFRα mutations is shown in Table 1. The activity of the besylate salt of the present invention against these kinases is also displayed in FIGS. 1, 2, 3, and 4.

TABLE 1

| Kinase | IC50 (nM) |
|---|---|
| PDGFRα | 1.51 |
| PDGFRα-D842V | 1.48 |
| PDGFRα-T6741 | 1.29 |
| PDGFRα-V561D | 2.87 |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of inhibiting or reducing mutant PDGFRα tyrosine kinase activity or expression in a subject suffering from a proliferative disorder or proliferative disease comprising:
obtaining a tumor sample from the subject;
measuring expression of a mutated PDGFRα or a constitutively active PDGFRα mutant; and
administering to the subject a therapeutically effective amount of crenolanib or a pharmaceutically acceptable salt thereof wherein the crenolanib or salt thereof reduces the proliferative disorder burden or prevents proliferative disease progression.

2. The method of claim 1, wherein the subject is at least one of: relapsed/refractory to a prior tyrosine kinase inhibitor; the subject has been provided a prior tyrosine kinase inhibitor selected from imatinib or avapritinib; or the subject has a PDGFRα mutation that is resistant to avapritinib.

3. The method of claim 1, wherein the PDGFRα mutation is selected from a missense mutation at D68, D135, D173, E229, C235, E262, T276, E289, K385, T440, A498, V561, R588, G608, N659, E675, Y676, S695, G741, G829, R841, I843, D846, Y849, N848, A1014, or D1071 present alone or in combination with a D842 missense mutation; or the PDGFRα mutation is selected from inframe deletions or insertions at amino acid residues R560-V561, R560-S564, E561-R562, S566-571, I843, D842-H845, or H845-5847 present alone or in combination with a D842 missense mutation.

4. The method of claim 1, wherein the proliferative disorder is selected from at least one of a gastrointestinal stromal tumor, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy.

5. The method of claim 1, wherein the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day.

6. The method of claim 1, wherein the therapeutically effective amount of crenolanib or the pharmaceutically acceptable salt thereof is administered at least one of:
administered continuously, intermittently, systemically, or locally;
administered orally, intravenously, or intraperitoneally;
administered up to three times or more a day for as long as the subject is in need of treatment for the proliferative disorder; or the therapeutically effective amount of crenolanib; or
administered at least one of sequentially or concomitantly, with another pharmaceutical agent.

7. The method of claim 1, wherein the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, or crenolanib succinate.

8. A method of inhibiting or reducing mutant PDGFRα tyrosine kinase activity or expression in a subject suffering from a proliferative disorder or proliferative disease comprising;
identifying that the subject discontinued a first tyrosine kinase inhibitor therapy due to toxicity or toxicities;
obtaining a tumor sample from the subject;
measuring expression of a mutated PDGFRα or a constitutively active PDGFRα mutant; and
if the subject has the mutated PDGFRα or constitutively active PDGFRα mutant, administering to the subject a therapeutically effective amount of crenolanib or a pharmaceutically acceptable salt thereof wherein the crenolanib or salt thereof reduces the proliferative disorder burden or prevents proliferative disease progression.

9. The method of claim 8, wherein the toxicity or toxicities requiring discontinuation of the first tyrosine kinase inhibitor therapy include one or more of:
intracranial hemorrhage, central nervous system toxicity, fatigue, abdominal pain, vomiting, sepsis, anemia, acute kidney injury, and encephalopathy;
wherein the intracranial hemorrhage includes one or more of subdural hematoma, cerebral hemorrhage, or other intracranial hemorrhage;
wherein the central nervous system toxicity includes one or more of cognitive impairment, dizziness, sleep disorders, mood disorders, and hallucinations; or
wherein the cognitive impairment includes one or more of memory impairment, cognitive disorder, confused state, disturbance in attention, amnesia, mental impairment, mental status changes, dementia, abnormal thinking, mental disorders, and retrograde amnesia.

10. The method of claim 8, wherein the proliferative disorder is selected from at least one of a gastrointestinal stromal tumor, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy.

11. The method of claim 8, wherein the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day.

12. The method of claim 8, wherein the therapeutically effective amount of crenolanib or the pharmaceutically acceptable salt thereof is administered at least one of:
administered continuously, intermittently, systemically, or locally;
administered orally, intravenously, or intraperitoneally;
administered up to three times or more a day for as long as the subject is in need of treatment for the proliferative disorder; or the therapeutically effective amount of crenolanib; or
administered at least one of sequentially or concomitantly, with another pharmaceutical agent.

13. The method of claim 8, wherein the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, and crenolanib succinate.

14. A method for treating a patient is suffering from a proliferative disorder or a proliferative disease, the method comprising the steps of:
determining whether the patient has increased PDGFRα tyrosine kinase activity by:
obtaining or having obtained a biological sample from the patient; and
performing or having performed an assay on the biological sample to determine if the patient has a gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity;
treating the patient with a first tyrosine kinase inhibitor (TKE); and
if the patient experiences a toxicity or toxicities to the first TKI and the patient has the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, then discontinue administering the first TKI and internally administering crenolanib to the patient in an effective amount, or
if the patient does not experience a toxicity or toxicities to the first TKI and the patient has the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, but the proliferative disorder or a proliferative disease progresses then discontinue administering the first TKI and internally administering crenolanib to the patient in an effective amount, or
if the patient has the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, and has proliferative disorder or proliferative disease progression then internally administering crenolanib to the patient in an effective amount,
wherein a risk of toxicity, toxicities or proliferative disorder or proliferative disease progression for a patient having the gene mutation in the PDGFRα tyrosine kinase activity, a change in the metabolic activity of the PDGFRα tyrosine kinase activity, overexpression of the PDGFRα tyrosine kinase, or a chance in the phenotype or genotype of the PDGFRα tyrosine kinase activity, is lower following the internal administration of crenolanib.

15. The method of claim 14, wherein the toxicity or toxicities requiring discontinuation of the first tyrosine kinase inhibitor therapy include one or more of intracranial hemorrhage, central nervous system toxicity, fatigue, abdominal pain, vomiting, sepsis, anemia, acute kidney injury, and encephalopathy.

16. The method of claim 14, wherein the toxicity or toxicities requiring discontinuation of the first tyrosine kinase inhibitor therapy include one or more of:
intracranial hemorrhage, central nervous system toxicity, fatigue, abdominal pain, vomiting, sepsis, anemia, acute kidney injury, and encephalopathy;
wherein the intracranial hemorrhage includes one or more of subdural hematoma, cerebral hemorrhage, or other intracranial hemorrhage;
wherein the central nervous system toxicity includes one or more of cognitive impairment, dizziness, sleep disorders, mood disorders, and hallucinations; or
wherein the cognitive impairment includes one or more of memory impairment, cognitive disorder, confused state, disturbance in attention, amnesia, mental impairment, mental status changes, dementia, abnormal thinking, mental disorders, and retrograde amnesia.

17. The method of any one of claims 26 to 30, wherein the proliferative disorder is selected from at least one of a gastrointestinal stromal tumor, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy.

18. The method of claim 14, wherein the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day.

19. The method of claim 14, wherein the therapeutically effective amount of crenolanib or the pharmaceutically acceptable salt thereof is administered at least one of: continuously, intermittently, systemically, or locally; or wherein the therapeutically effective amount of crenolanib or the pharmaceutically acceptable salt thereof is administered orally, intravenously, or intraperitoneally.

20. The method of claim 14, wherein the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, and crenolanib succinate.

21. The method of claim 14, wherein the therapeutically effective amount of crenolanib or the pharmaceutically acceptable salt thereof is at least one of:
administered up to three times or more a day for as long as the subject is in need of treatment for the proliferative disorder; or
administered at least one of sequentially or concomitantly, with another pharmaceutical agent.

22. The method of claim 14, wherein the PDGFRα mutation is selected from at least one of: a missense mutation at D68, D135, D173, E229, C235, E262, T276, E289, K385, T440, A498, V561, R588, G608, N659, E675, Y676, S695, G741, G829, R841, I843, D846, Y849, N848, A1014, or D1071 present alone or in combination with a D842 missense mutation; or the PDGFRα mutation is selected from inframe deletions or insertions at amino acid residues R560-V561, R560-S564, E561-R562, S566-571, I843, D842-H845, or H845-S847 present alone or in combination with a D842 missense mutation.
